SEQUENCE LISTING (23 sequences)

SEQ ID NO: 1 - DNA, 2334 bp, Artificial Sequence
  pBBR1 origin of replication
tgcttgagactggccgccacgttgcccattttcgccagcttcttgcatcgcatgatcgcgtatgccgccatgcctgcccctcccttttggtgtccaaccggctcgacgggggcagcgcaaggcggtgcctccggcgggccactcaatgcttgagtatactcactagactttgcttcgcaaagtcgtgaccgcctacggcggctgcggcgccctacgggcttgctctccgggcttcgccctgcgcggtcgctgcgctcccttgccagcccgtggatatgtggacgatggccgcgagcggccaccggctggctcgcttcgctcggcccgtggacaaccctgctggacaagctgatggacaggctgcgcctgcccacgagcttgaccacagggattgcccaccggctacccagccttcgaccacatacccaccggctccaactgcgcggcctgcggccttgccccatcaatttttttaattttctctggggaaaagcctccggcctgcggcctgcgcgcttcgcttgccggttggacaccaagtggaaggcgggtcaaggctcgcgcagcgaccgcgcagcggcttggccttgacgcgcctggaacgacccaagcctatgcgagtgggggcagtcgaaggcgaagcccgcccgcctgccccccgagcctcacggcggcgagtgcgggggttccaagggggcagcgccaccttgggcaaggccgaaggccgcgcagtcgatcaacaagccccggaggggccactttttgccggagggggagccgcgccgaaggcgtgggggaaccccgcaggggtgcccttctttgggcaccaaagaactagatatagggcgaaatgcgaaagacttaaaaatcaacaacttaaaaaaggggggtacgcaacagctcattgcggcaccccccgcaatagctcattgcgtaggttaaagaaaatctgtaattgactgccacttttacgcaacgcataattgttgtcgcgctgccgaaaagttgcagctgattgcgcatggtgccgcaaccgtgcggcaccctaccgcatggagataagcatggccacgcagtccagagaaatcggcattcaagccaagaacaagcccggtcactgggtgcaaacggaacgcaaagcgcatgaggcgtgggccgggcttattgcgaggaaacccacggcggcaatgctgctgcatcacctcgtggcgcagatgggccaccagaacgccgtggtggtcagccagaagacactttccaagctcatcggacgttctttgcggacggtccaatacgcagtcaaggacttggtggccgagcgctggatctccgtcgtgaagctcaacggccccggcaccgtgtcggcctacgtggtcaatgaccgcgtggcgtggggccagccccgcgaccagttgcgcctgtcggtgttcagtgccgccgtggtggttgatcacgacgaccaggacgaatcgctgttggggcatggcgacctgcgccgcatcccgaccctgtatccgggcgagcagcaactaccgaccggccccggcgaggagccgcccagccagcccggcattccgggcatggaaccagacctgccagccttgaccgaaacggaggaatgggaacggcgcgggcagcagcgcctgccgatgcccgatgagccgtgttttctggacgatggcgagccgttggagccgccgacacgggtcacgctgccgcgccggtagcacttgggttgcgcagcaacccgtaagtgcgctgttccagactatcggctgtagccgcctcgccgccctataccttgtctgcctccccgcgttgcgtcgcggtgcatggagccgggccacctcgacctgaatggaagccggcggcacctcgctaacggattcaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtcggcctattggttaaaaaatgagctgatttaacaaaaatttaacgcgaattttaacaaaatattaacgcttacaatttccattcgccattcaggctgcgcaactgttgggaagggcgat

SEQ ID NO: 2 - DNA, 1511 bp, Artificial Sequence
  Modified pWV01 origin of replication
cgattttttattaaaacgtctcaaaatcgtttctgagacgttttagcgtttatttcgtttagttatcggcataatcgttaaaacaggcgttatcgtagcgtaaaagcccttgagcgtagcgtggctttgcagcgaagatgttgtctgttagattatgaaagccgatgactgaatgaaataataagcgcagcgcccttctatttcggttggaggaggctcaagggagtatgagggaatgaaattccctcatgggtttgattttaaaaattgcttgcattttgccgagcggtagcgctggaaaatttttgaaaaaaatttggaatttggaaaaaaatggggggaaaggaagcgaattttgcttccgtactacgaccccccattaagtgccgagtgccaatttttgtgccaaaaacgctctatcccaactggctcaagggtttaaggggtttttcaatcgccaacgaatcgccaacgttttcgccaacgttttttataaatctatatttaagtagctttattgttgtttttatgattacaaagtgatacactaactttataaaattatttgattggagttttttaaatggtgatttcagaatcgaaaaaaagagttatgatttctctgacaaaagagcaagataaaaaattaacagatatggcgaaacaaaaaggtttttcaaaatctgcggttgcggcgttagctatagaagaatatgcaagaaaggaatcagaacaaaaaaaataagcgaaagctcgcgtttttagaaggatacgagttttcgctacttgtttttgataaggtaattatatcatggctattaaaaatactaaagctagaaattttggatttttattatatcctgactcaattcctaatgattggaaagaaaaattagagagtttgggcgtatctatggctgtcagtcctttacacgatatggacgaaaaaaaagataaagatacatggaatagtagtgatgttatacgaaatggaaagcactataaaaaaccacactatcacgttatatatattgcacgaaatcctgtaacaatagaaagcgttaggaacaagattaagcgaaaattggggaatagttcagttgctcatgttgagatacttgattatatcaaaggttcgtatgaatatttgactcatgaatcaaaggacgctattgctaagaataaacatatatacgacaaaaaagatattttgaacattaatgattttgatattgaccgctatataacacttgatgaaagccaaaaaagagaattgaagaatttacttttagatatagtggatgactataatttggtaaatacaaaagatttaatggcttttattcgccttaggggagcggagtttggaattttaaatacgaatgatgtaaaagatattgtttcaacaaactctagcgcctttagattatggtttgagggcaattatcagtgtggatatagagcaagttatgcaaaggttcttgatgctgaaacgggggaaataaaatgac

SEQ ID NO: 3 - DNA, 39 bp, Artificial Sequence
  Terminator sequence 1
aaaaaaaaaggcctgcgattacccgcaggcctttttttt

SEQ ID NO: 4 - DNA, 47 bp, Artificial Sequence
  Terminator sequence 2
tagcataaccccttggggcctctaaacgggtcttgaggggttttttg

SEQ ID NO: 5 - DNA, 28 bp, Artificial Sequence
  Terminator sequence 3
aaaaaaaagcccgctcattaggcgggct

SEQ ID NO: 6 - DNA, 30 bp, Artificial Sequence
  Terminator sequence 4
acagaaaagcccgcctttcggcgggctttg

SEQ ID NO: 7 - DNA, 77 bp, Artificial Sequence
  Promotor sequence
tgttgacaattaatcatcggctcgtataatgtgtggaattgtgagcggataacaatttcacacaggaaacagaattc

SEQ ID NO: 8 - DNA, 6420 bp, Artificial Sequence
  Shuttle vector, whole sequence
cctgtgcaaggagtacttcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagaattccatatggatatcgagctcggtacccggggatcctctagagtcgacctgcaggcatgcaagcttctcgagaaaaaaaagcccgctcattaggcgggctaaaaaaaaagcccgcctaatgagcgggcttttttttacagaaaagcccgcctttcggcgggctttgagcggccgcgtcattttatttcccccgtttcagcatcaagaacctttgcataacttgctctatatccacactgataattgccctcaaaccataatctaaaggcgctagagtttgttgaaacaatatcttttacatcattcgtatttaaaattccaaactccgctcccctaaggcgaataaaagccattaaatcttttgtatttaccaaattatagtcatccactatatctaaaagtaaattcttcaattctcttttttggctttcatcaagtgttatatagcggtcaatatcaaaatcattaatgttcaaaatatcttttttgtcgtatatatgtttattcttagcaatagcgtcctttgattcatgagtcaaatattcatacgaacctttgatataatcaagtatctcaacatgagcaactgaactattccccaattttcgcttaatcttgttcctaacgctttctattgttacaggatttcgtgcaatatatataacgtgatagtgtggttttttatagtgctttccatttcgtataacatcactactattccatgtatctttatcttttttttcgtccatatcgtgtaaaggactgacagccatagatacgcccaaactctctaatttttctttccaatcattaggaattgagtcaggatataataaaaatccaaaatttctagctttagtatttttaatagccatgatataattaccttatcaaaaacaagtagcgaaaactcgtatccttctaaaaacgcgagctttcgcttattttttttgttctgattcctttcttgcatattcttctatagctaacgccgcaaccgcagattttgaaaaacctttttgtttcgccatatctgttaattttttatcttgctcttttgtcagagaaatcataactctttttttcgattctgaaatcaccatttaaaaaactccaatcaaataattttataaagttagtgtatcactttgtaatcataaaaacaacaataaagctacttaaatatagatttataaaaaacgttggcgaaaacgttggcgattcgttggcgattgaaaaaccccttaaacccttgagccagttgggatagagcgtttttggcacaaaaattggcactcggcacttaatggggggtcgtagtacggaagcaaaattcgcttcctttccccccatttttttccaaattccaaatttttttcaaaaattttccagcgctaccgctcggcaaaatgcaagcaatttttaaaatcaaacccatgagggaatttcattccctcatactcccttgagcctcctccaaccgaaatagaagggcgctgcgcttattatttcattcagtcatcggctttcataatctaacagacaacatcttcgctgcaaagccacgctacgctcaagggcttttacgctacgataacgcctgttttaacgattatgccgataactaaacgaaataaacgctaaaacgtctcagaaacgattttgagacgttttaataaaaaatcggcggccgcaaaaaaaaaggcctgcgattacccgcaggccttttttttgctcttcgcgactagttttacaaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaatttttatctaaagtgaatttaggaggcttacttgtctgctttcttcattagaatcaatcccacgtgagatctttttttaaaagtcaatattactgtaacataaatatatattttaaaaatatcccactttatccaattttcgtttgttgaactaatgggtgctttagttgaagatgcttgagactggccgccacgttgcccattttcgccagcttcttgcatcgcatgatcgcgtatgccgccatgcctgcccctcccttttggtgtccaaccggctcgacgggggcagcgcaaggcggtgcctccggcgggccactcaatgcttgagtatactcactagactttgcttcgcaaagtcgtgaccgcctacggcggctgcggcgccctacgggcttgctctccgggcttcgccctgcgcggtcgctgcgctcccttgccagcccgtggatatgtggacgatggccgcgagcggccaccggctggctcgcttcgctcggcccgtggacaaccctgctggacaagctgatggacaggctgcgcctgcccacgagcttgaccacagggattgcccaccggctacccagccttcgaccacatacccaccggctccaactgcgcggcctgcggccttgccccatcaatttttttaattttctctggggaaaagcctccggcctgcggcctgcgcgcttcgcttgccggttggacaccaagtggaaggcgggtcaaggctcgcgcagcgaccgcgcagcggcttggccttgacgcgcctggaacgacccaagcctatgcgagtgggggcagtcgaaggcgaagcccgcccgcctgccccccgagcctcacggcggcgagtgcgggggttccaagggggcagcgccaccttgggcaaggccgaaggccgcgcagtcgatcaacaagccccggaggggccactttttgccggagggggagccgcgccgaaggcgtgggggaaccccgcaggggtgcccttctttgggcaccaaagaactagatatagggcgaaatgcgaaagacttaaaaatcaacaacttaaaaaaggggggtacgcaacagctcattgcggcaccccccgcaatagctcattgcgtaggttaaagaaaatctgtaattgactgccacttttacgcaacgcataattgttgtcgcgctgccgaaaagttgcagctgattgcgcatggtgccgcaaccgtgcggcaccctaccgcatggagataagcatggccacgcagtccagagaaatcggcattcaagccaagaacaagcccggtcactgggtgcaaacggaacgcaaagcgcatgaggcgtgggccgggcttattgcgaggaaacccacggcggcaatgctgctgcatcacctcgtggcgcagatgggccaccagaacgccgtggtggtcagccagaagacactttccaagctcatcggacgttctttgcggacggtccaatacgcagtcaaggacttggtggccgagcgctggatctccgtcgtgaagctcaacggccccggcaccgtgtcggcctacgtggtcaatgaccgcgtggcgtggggccagccccgcgaccagttgcgcctgtcggtgttcagtgccgccgtggtggttgatcacgacgaccaggacgaatcgctgttggggcatggcgacctgcgccgcatcccgaccctgtatccgggcgagcagcaactaccgaccggccccggcgaggagccgcccagccagcccggcattccgggcatggaaccagacctgccagccttgaccgaaacggaggaatgggaacggcgcgggcagcagcgcctgccgatgcccgatgagccgtgttttctggacgatggcgagccgttggagccgccgacacgggtcacgctgccgcgccggtagcacttgggttgcgcagcaacccgtaagtgcgctgttccagactatcggctgtagccgcctcgccgccctataccttgtctgcctccccgcgttgcgtcgcggtgcatggagccgggccacctcgacctgaatggaagccggcggcacctcgctaacggattcaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtcggcctattggttaaaaaatgagctgatttaacaaaaatttaacgcgaattttaacaaaatattaacgcttacaatttccattcgccattcaggctgcgcaactgttgggaagggcgatagatcttagcataaccccttggggcctctaaacgggtcttgaggggttttttgccactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttgacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcaacgtaaatgccgcttcgccttcggcgcgcgaattgcaagctgatccggcggagcttatcgactgcacggtggctgtggtatgg

SEQ ID NO: 9 - DNA, 751 bp, Artificial Sequence
  Chloramphenicol acetyltransferase cassette (PmlI to SapI)
cacgtgggattgattctaatgaagaaagcagacaagtaagcctcctaaattcactttagataaaaatttaggaggcatatcaaatgaactttaataaaattgatttagacaattggaagagaaaagagatatttaatcattatttgaaccaacaaacgacttttagtataaccacagaaattgatattagtgttttataccgaaacataaaacaagaaggatataaattttaccctgcatttattttcttagtgacaagggtgataaactcaaatacagcttttagaactggttacaatagcgacggagagttaggttattgggataagttagagccactttatacaatttttgatggtgtatctaaaacattctctggtatttggactcctgtaaagaatgacttcaaagagttttatgatttatacctttctgatgtagagaaatataatggttcggggaaattgtttcccaaaacacctatacctgaaaatgctttttctctttctattattccatggacttcatttactgggtttaacttaaatatcaataataatagtaattaccttctacccattattacagcaggaaaattcattaataaaggtaattcaatatatttaccgctatctttacaggtacatcattctgtttgtgatggttatcatgcaggattgtttatgaactctattcaggaattgtcagataggcctaatgactggcttttgtaaaactagtcgcgaagagc

SEQ ID NO: 10 - DNA, 1255 bp, Artificial Sequence
  lacI cassette (EcoNI to Stop)
ccttgcacaggccataccacagccaccgtgcagtcgataagctccgccggatcagcttgcaattcgcgcgccgaaggcgaagcggcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggacatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtg

SEQ ID NO: 11 - DNA, 22 bp, Artificial Sequence
  lac operator cassette
gaattgtgagcggataacaatt

SEQ ID NO: 12 - DNA, 19 bp, Artificial Sequence
  Primer sequence pPR-HisT_1
caccgtgcagtcgataagc

SEQ ID NO: 13 - DNA, 18 bp, Artificial Sequence
  Primer sequence pPR-HisT_2
gctgtggtatggcctgtg

SEQ ID NO: 14 - DNA, 23 bp, Artificial Sequence
  Primer sequence GA_pPR_1
cacattcaccaccctgaattgac

SEQ ID NO: 15 - DNA, 26 bp, Artificial Sequence
  Primer sequence GA_pPR_2
gatctcacgtgggattgattctaatg

SEQ ID NO: 16 - DNA, 60 bp, Artificial Sequence
  Primer sequence GA_pPR_3
tagcataaccccttggggcctctaaacgggtcttgaggggttttttgtcactgcccgctt

SEQ ID NO: 17 - DNA, 23 bp, Artificial Sequence
  Primer sequence GA_pPR_4
gtcaattcagggtggtgaatgtg

SEQ ID NO: 18 - DNA, 26 bp, Artificial Sequence
  Primer sequence GA_pPR_5
cattagaatcaatcccacgtgagatc

SEQ ID NO: 19 - DNA, 60 bp, Artificial Sequence
  Primer sequence GA_pPR_6
caaaaaacccctcaagacccgtttagaggccccaaggggttatgctaagatctatcgccc

SEQ ID NO: 20 - DNA, 55 bp, Artificial Sequence
  Primer sequence pWV01_opt_1
aaggaaaaaagcggccgccgattttttattaaaacgtctcaaaatcgtttctgag

SEQ ID NO: 21 - DNA, 46 bp, Artificial Sequence
  Primer sequence pWV01_opt_2
aaggaaaaaagcggccgcgtcattttatttcccccgtttcagcatc

SEQ ID NO: 22 - DNA, 24 bp, Artificial Sequence
  Primer sequence GFP_for
atggtccaaactagttcgaagatc

SEQ ID NO: 23 - DNA, 49 bp, Artificial Sequence
  Primer sequence GFP_His6_rev
gctctagactaatgatgatggtgatgatgtttgtagggctcatccatgc

(19) United States
(12) Patent Application Publication
(10) Pub. No.: US 2015/0072898 A1
(43) Pub. Date: Mar. 12, 2015

(54) Broad Host Range Expression Vector for Diverse Prokaryotes

(71) Applicant: WESTFAELISCHE WILHELMS-UNIVERSITAET MUENSTER, MUENSTER (DE)

(72) Inventors: Bruno Moerschbacher, Muenster (DE); Stephan Kolkenbrock, Duesseldorf (DE)

(21) Appl. No.: 14/483,185

(22) Filed: Sep. 11, 2014

(30) Foreign Application Priority Data
Sep. 11, 2013 (EP) 13 183 909.4

(51) Int. Cl. C12N 15/66

(52) U.S. Cl. CPC C12N 15/66
USPC 506/26; 435/320.1; 435/252.33; 435/91.1; 435/252.34; 435/252.31

(57) ABSTRACT

The invention relates to a synthetic nucleic acid molecule for expressing at least one nucleotide sequence of interest in at least one prokaryotic host cell, comprising, amongst others, at least one replication module comprising at least one replication cassette for promoting replication of the nucleic acid molecule in Gram-negative organisms and at least one replication cassette for promoting replication of the nucleic acid molecule in Gram-positive organisms, and at least one expression module for promoting expression of the nucleotide sequence of interest in the host cell, wherein each module is flanked at both ends by at least one unique restriction site. The invention further concerns a method for producing a shuttle vector comprising several modules, wherein said shuttle vector is designed by selecting each of said modules such that the vector is optimized for its intended use.

### FIELD OF THE INVENTION

The invention relates to a synthetic nucleic acid molecule for expressing at least one nucleotide sequence of interest in at least one prokaryotic host cell, comprising at least one promoter sequence and at least one cloning site for inserting the nucleotide sequence of interest, wherein the cloning site is located downstream of the promoter sequence. The invention also relates to a method for producing a shuttle vector, said vector comprising at least one replication module comprising at least one replication cassette for promoting replication of a nucleic acid molecule in Gram-negative organisms and at least one replication cassette for promoting replication of a nucleic acid molecule in Gram-positive organisms, at least one expression module for promoting expression of a nucleotide sequence of interest in a host cell, and at least one resistance module for providing the host cell with antibiotic resistance.

### BACKGROUND AND INTRODUCTION TO THE INVENTION

The heterologous expression of genes in prokaryotes is challenging, especially if the genes originate from a distant host or if the source is uncertain, such as a metagenomic expression library. Many vectors have been developed based on broad host range origins of replication, but these all focus on either Gram(+) or Gram(−) prokaryotes.

Escherichia coli is the working horse in biotechnology for decades. But with the shifting focus in biotechnology to functional metagenomics, the expression of environmental DNA (eDNA) in E. coli becomes a bottleneck. One may be able to optimize DNA sequences for the needs of E. coli and to generate novel E. coli strains, but this does not apply for the establishment of expression libraries with unknown DNA sequences, such as eDNA. That prevents us from harnessing the enormous biotechnological potential of genomes from uncultured microorganisms. However some approaches have already been followed to circumvent E. coli as an expression host, such as the establishment of a metagenomic expression library in Cupriavidus metaffidurans, Streptomyces spp., and Pseudomonas fluorescens.

Although some of these approaches are based on broad-host range expression vectors, the number of hosts is very limited due to the focus on either Gram(+) or Gram(−) organisms and the fact that most vectors replicate preferentially either in Gram(+) or Gram(−) organisms.

Today's broad host vectors are mainly based on IncaP, RK2 or rolling circle replicating (RCR) plasmids like pCI411. A very frequently used RCR-plasmid is pGK12 from Kluyveromyces lactis CBS 2359, which can replicate in E. coli and mainly in Gram(+) organisms like Bacillus subtilis, Borrelia burgdorferi and Lactococcus lactis. Although its RCR origin is used in over 20 shuttle vectors, it has numerous disadvantages like its big size and instabilities in any host. Due to this poor performance it was never widely adopted and alternatives are of great interest.

Accordingly, there is a need in the art for an expression vector for establishing expression libraries with unknown DNA sequences, such as environmental DNA. There is also a need in the art for a broad-host range expression vector which replicates in both Gram(+) and Gram(−) organisms.

### SUMMARY OF THE INVENTION

The invention is directed at a synthetic nucleic acid molecule comprising at least one replication module comprising at least one replication cassette for promoting replication of the nucleic acid molecule in Gram-negative organisms and at least one replication cassette for promoting replication of the nucleic acid molecule in Gram-positive organisms, at least one expression module for promoting expression of the nucleotide sequence of interest in the host cell, and at least one resistance module for providing the host cell with antibiotic resistance, wherein the at least one replication module, the at least one expression module and the at least one resistance module are each flanked at both ends by at least one unique restriction site. The nucleic acid molecule according to the invention may represent a fully synthetic expression vector based on/comprising different origins of replication so as to allow for using various Gram(+) and Gram(−) hosts at the same time for expression. Thus, it is easily possible to change the cloning systems without the need of additional cloning. This also makes it easy to generate environmental DNA (eDNA) expression libraries for the functional screening in diverse hosts without focusing on either Gram(+) or Gram(−) organisms. Thus, the tool described herein may allow for the identification of novel biocatalysts, which, so far, were not functional in the limited number of vector compatible hosts. Moreover, the modular design of the nucleic acid molecule according to the invention allows for constructing diverse expression vectors which are each optimized for their intended use. To this end, each module of the nucleic acid molecule is flanked at both ends by at least one unique restriction site so that each module may be replaced by another module having a different function and/or effect. This measure makes it easy to combine different modules such that the resulting expression vector is optimally adapted to its intended application.

In an embodiment of the present invention the replication cassette for Gram-negative organisms can, for example, comprise a pBBR1 origin of replication. For example, the replication cassette for Gram-negative organisms can comprise the nucleotide sequence according to SEQ ID NO: 1.

In an embodiment of the present invention the replication cassette for Gram-positive organisms can comprise a pWV01 origin of replication, for example, a modified pWV01 origin of replication. In an embodiment of the present invention, the replication cassette for Gram-positive organisms can, for example, comprise the nucleotide sequence according to SEQ ID NO: 2. This sequence represents a modified pWV01 which is optimized for use in the nucleic acid molecule according to the invention.

The pWV01 origin of replication of Lactococcus lactis subsp. cremoris Wg2 is much smaller than the RCR origin of pGK12 and seems to have a higher performance in terms of copy number and stability. The pBBR1 origin of replication of Bordetella bronchiseptica S87 is widely used and compatible with IncP, IncQ and IncW group plasmids as well as with ColE1 and p15A containing plasmids. While the pBBR1 mode of replication is unclear, pWV01 replicates via the rolling circle mechanism. However, it is surprising that these origins are compatible and can be placed on the same vector without any interference. It is therefore an advantageous aspect of the invention that pBBR1 and an optimized pWV01 may be combined on the same completely synthesized vector.

In an embodiment of the present invention the unique restriction site can, for example, be selected from the group consisting of BglII, NotI, PmlI, and SapI. However, it is also possible to use other unique restriction sites as long as the modular character of the nucleic acid molecule according to the invention is maintained.

The nucleic acid molecule according to the present invention can, for example, further comprise at least one transcription termination sequence located downstream of the cloning site, for example, a transcription termination sequence selected from the group consisting of SEQ ID NO: 3 (new_Ter), SEQ ID NO: 4 (T7_Ter), SEQ ID NO: 5 (trpA_Ter), and SEQ ID NO: 6 (t500_Ter).

While an initially developed vector was able to replicate solely in Escherichia coli Stbl2, a specialized strain used to house unstable inserts containing repetitive sequences, deletion of some of its terminator sequences and having the common E. coli strain DH5α select which of the remaining terminators were compatible with stable replication and thus the maintenance of a full-length vector, resulted in the expression vector according to the invention. Surprisingly, this strain not only preferred a specific set of terminators (SEQ ID NO: 4, SEQ ID NO: 5, and SEQ ID NO: 6) but also generated a completely novel one (SEQ ID NO: 3).

In an embodiment of the present invention the expression module can, for example, comprise a promoter sequence. For example, the promoter sequence can comprise a Ptac promoter sequence (SEQ ID NO: 7).

In an embodiment of the present invention the expression module can, for example, comprise at least one regulatory sequence, for example, a lacI cassette and a lac operator sequence. In an embodiment of the present invention, the expression module can, for example, comprise the cloning site, for example, a multiple cloning site.

In an embodiment of the present invention the resistance module can, for example, comprise a chloramphenicol acetyl transferase (CAT) resistance cassette.

For example, the synthetic nucleic acid molecule according to the invention can, comprise at least one nucleotide sequence selected from the group consisting of:
a) a nucleotide sequence which comprises the nucleotide sequences of SEQ ID NO: 1, SEQ ID NO: 2, SEQ ID NO: 3, SEQ ID NO: 4, SEQ ID NO: 5, SEQ ID NO: 6, and SEQ ID NO: 7;
b) a nucleotide sequence according to SEQ ID NO: 8;
c) a nucleotide sequence, the complementary strand of which hybridizes with the nucleotide sequences of a) or b) under stringent conditions;
d) a nucleotide sequence which has at least 90% or 95%, identity with the nucleotide sequence of a), b) or c); and
e) a nucleotide sequence which corresponds to the complementary strand of the nucleotide sequence of a) to d).

The invention further concerns a prokaryotic cell including the nucleic acid molecule according to the invention as described above.

The invention also relates to a cell culture comprising at least one cell according to the invention.

Moreover, the invention relates to a polypeptide, produced by expression of a nucleotide sequence of interest in a prokaryotic host cell using the nucleic acid molecule according to the invention as described above.

A further aspect of the invention is the use of the nucleic acid molecule according to the invention as described above for heterologous expression of metagenomic expression libraries in at least one prokaryotic host cell, for example, for functional screening of environmental expression libraries. The nucleic acid molecule according to the invention can, for example, also be used for generating cDNA expression libraries of eukaryotic genes or introducing recombination cassettes for promoting specific knockouts or accelerating chromosomal localization.

Another aspect of the invention relates to a method for expressing at least one nucleotide sequence of interest in at least one prokaryotic host cell, said method comprising the following steps:
inserting the nucleotide sequence of interest into a cloning site of the nucleic acid molecule according to the invention as described above;
subsequently, introducing the nucleic acid molecule into a host cell to obtain a modified host cell;
cultivating the modified host cell under conditions that allow expression of the nucleotide sequence of interest.

For example, the nucleic acid molecule may be heterologously expressed in at least one prokaryotic host cell.

In an embodiment of said method, the nucleotide sequence of interest can, for example, be part of a metagenomic library. According to the invention environmental DNA (eDNA) expression libraries can be easily generated and used for the functional screening in diverse hosts without focusing on either Gram(+) or Gram(−) organisms.

The invention is also directed at a method for producing a shuttle vector, said vector comprising at least one replication module comprising at least one replication cassette for promoting replication of a nucleic acid molecule in Gram-negative organisms and at least one replication cassette for promoting replication of a nucleic acid molecule in Gram-positive organisms, at least one expression module for promoting expression of a nucleotide sequence of interest in a host cell, and at least one resistance module for providing the host cell with antibiotic resistance, wherein said shuttle vector is obtained by assembling each of said modules, preferably such that the vector is optimized for its intended use. According to this aspect of the invention, different modules may be combined such that the resulting expression vector is perfectly adapted to its intended application. For example, each module of the vector may be flanked at both ends by at least one unique restriction site so that each module can be easily replaced by another module having a different function and/or effect.

### BRIEF DESCRIPTION OF THE DRAWINGS

The invention is further exemplarily described in detail with reference to the figures.

FIG. 1: Generation of pPolyREP. This figure shows the structure of an initially constructed vector called pPolyREP comprising four major modules: the replication modules pBBR1 (SEQ ID NO: 1) and pWV01 (B); the resistance module containing a chloramphenicol acetyltransferase cassette (CAT) (SEQ ID NO: 9) (C); and the expression module containing a lacI cassette (SEQ ID NO: 10), a Ptac (SEQ ID NO: 7) promoter, a lac operator (SEQ ID NO: 11) and a multiple cloning site (D). This initially produced vector already has the modular character according to the invention, i.e. each module is flanked by unique restriction sites (B, C, D) and can be removed or replaced easily.

FIG. 2: Analysis of pPolyREP (A) and the derivatives pPR_pBBR1 (B) and pPR_pWV01 (C). This figure shows a gel electrophoretic analysis of the initially constructed vector pPolyREP. As becomes apparent, each origin of replication can be removed easily using either NotI, which removes pWV01 and generates pPR_pBBR1, or BglII, which removes pBBR1 and generates pPR_pWV01 (See also FIG. 1). While each origin can be removed from the full-length vector pPolyREP (NotI, 5216+2187 bp; BglII, 4961+2442 bp, A), only the remaining origins in the derived vectors can be removed (BglII, 2774+2442 bp, B; NotI, 2774+2187 bp, C). Isolation of the derivatives from E. coli Stbl2 clearly demonstrates the functionality of both individual origins. The NdeI and XhoI sites are part of the multiple cloning site and were used to linearize the vectors (7403 bp).

### M: GeneRuler 1 kb DNA Ladder, Thermo Scientific, Germany.

FIG. 3: DNA sequences and calculated secondary structures of transcription terminators used. This figure shows the structure of the transcription terminators according to the invention. The upper lane represents the transcription terminators present in pPolyREP, which were replaced, modified or deleted in one embodiment of a vector according to the invention (pPolyREPII). The lower lane represents the set of terminators, which were used in the final vector pPolyREPII. The difference in the calculated Gibbs free energy between the original tR2 terminator and the newly-generated terminator (new_Ter; SEQ ID NO: 3) is almost half that of tR2. This reflects the presence of two point mutations, marked with arrows, which reduce the size of the terminator loop but produce a longer stem and thus make it stronger than the original. The structure and free Gibbs energy were calculated with the mfold program.

FIG. 4: Modification of pPolyREP to generate an improved vector according to the invention pPolyREPII. This figure shows the structures of the initially constructed vector pPolyREP and an improved vector according to the invention, pPolyREPII (SEQ ID NO: 8). To generate an optimized shuttle vector that can replicate in common E. coli strains such as DH5α, we removed the dual histidine terminators (his_Ter) and replaced the dual rrnB terminators (rrnB_Ter) with a T7 terminator (T7_Ter; SEQ ID NO: 4). Apparently, E. coli DH5α still experienced difficulties with the dual tR2 terminators (tR2_Ter) as the vector recovered from the transformed strains contained only a single copy of tR2_Ter, which was even modified to form a new terminator (new_Ter; SEQ ID NO: 3, FIG. 3). These optimizations made it possible to transform E. coli DH5α with the new vector, which was maintained in this host in its original form. Later, we also optimized the origin pWV01 by truncation and reversing its orientation (Modified pWV01; SEQ ID NO: 2). These optimizations led to the final version of an exemplary vector according to the invention, pPolyREPII.

FIG. 5: Analysis of the derivatives of pPolyREPII, pPolyREPII(+) and pPolyREPII(−) isolated from E. coli DH5α (A); and the structure of the original and new transcription terminators (B). This figure shows a gel electrophoretic analysis of derivatives of pPolyREPII, wherein pPolyREPII(+) and pPolyREPII(−) are derivatives of pPolyREPII containing only the pWV01 or pBBR1 origins, respectively. While pWV01 can be removed from pPolyREPII(+) with NotI (2459+1519 bp), this vector is linearized by BglII (3978 bp) due to the missing pBBR1 origin. Similarly, whereas pBBR1 can be removed from pPolyREPII(−) with BglII (2459+2442 bp), this vector is linearized by NotI (4901 bp) due to the missing pWV01 origin. The digestion of pPolyREPII(−) with BglII generates two fragments almost equal in size. Therefore we also digested pPolyREPII(−) with McsI in addition to BglII, which cuts pBBR1 into almost equal-sized fragments (1267+1175 bp) and leaves the second fragment of the BglII digest untouched (2459 bp).

### M: GeneRuler 1 kb DNA Ladder, Thermo Scientific, Germany.

FIG. 6: Expression studies with pPolyREPII and pPRII::GFP-His₆. This figure shows a SDS-PAGE (left) and corresponding western blot with chemiluminescence detection using Anti-Penta-His IgG1 HRP conjugate (Qiagen, Germany) (right) of the crude extracts of each host transformed with pPRII:GFP-His₆ (A). The band detected in the crude extracts of E. coli DH5α, P. putida KT2440 and B. subtilis 168 corresponds to the molecular mass of GFP-His₆ (˜29.6 kDa). Each host was transformed with the empty vector pPolyREPII (1) and pPRII:GFP-His₆ (2) and the synthesis of GFP-His₆ was observed on a transilluminator (Blue LED Illuminator, excitation wavelength of 470 nm; NIPPON Genetics EUROPE GmbH, Germany) (B).

FIG. 7: Segregational stability of pPolyREPII in various hosts. The number of resistant cell forming units after 0, 2, and 5 passages is expressed as the ratio relative to the initial number of cell forming units (A). To verify vector integrity, pPolyREPII was isolated from various hosts after 5 passages and compared to the restriction pattern obtained directly from E. coli DH5α without passaging (B). The isolated vector DNA was separately digested with NotI (4901+1519 bp) and BglII (3978+2442 bp). (M1: GeneRuler 1 kb DNA Ladder, Thermo Scientific, Germany; M2: 1 kb DNA Ladder, New England Biolabs, Germany; E.c., E. coli DH5α; P.p., P. putida KT2440; C.m., C. metaffidurans CH34; B.l., B. licheniformis DSM13; B.s., B. subtilis 168)

FIG. 8: Schematic representation of the pPolyREPII generation. Starting from the initial synthetic construct pPolyREP (A), the dual histidine terminator sequences (his_Ter) were removed, generating pPolyREP Δ2×his_Ter (B). By transforming the latter construct in E. coli DH5α the re-isolated vector was truncated to a 3852 bp vector (C), which was the template for the amplification of fragment 1. For the reconstruction of pPolyREP, fragments 2 and 3 were amplified from pPolyREP Δ2×his_Ter and used for the re-construction to pPolyREPII (D) via a Gibson assembly reaction. The resulting vector was further optimized by the truncation of the pWV01 origin of replication, resulting in the final shuttle vector according to the invention.

### DETAILED DESCRIPTION OF VARIOUS AND PREFERRED EMBODIMENTS OF THE INVENTION

The term “synthetic nucleic acid molecule” as used herein refers to a nucleic acid molecule that is constructed by joining nucleic acid molecules using laboratory methods or that is chemically or by other means synthesized or amplified. The term “synthetic nucleic acid molecule” includes but is not limited to molecules that are chemically or otherwise modified but can base pair with naturally occurring nucleic acid molecules or to molecules that result from the replication of those described above. The term “synthetic nucleic acid molecule” further includes but is not limited to recombinant nucleic acid molecules.

The term “recombinant nucleic acid molecules” as used herein refers to nucleic acid molecules constructed by laboratory methods of genetic recombination (such as molecular cloning) to bring together genetic material from different sources.

The term “flanked module” as used herein refers to a consecutive sequence of nucleotides, wherein at least one specific element (such as a restriction site) abuts this sequence at both ends of the sequence, i.e. at both the 3′ and the 5′ end.

The term “heterologous expression” as used herein refers to a process wherein a gene or gene fragment is expressed in a host organism which does not naturally have this gene or gene fragment.

The term “metagenomic library” as used herein refers to a pool of genetic material recovered directly from environmental material comprising largely unbiased samples of all genes from all the members of the material. “Environmental material” may include but is not limited to environmental DNA (eDNA).

The term “nucleic acid” as used herein refers to a polymeric molecule comprising a consecutive sequence of nucleotide monomers (“nucleotides”). A nucleic acid molecule according to the invention may include but is not limited to deoxyribonucleic acid (DNA), ribonucleic acid (RNA), and nucleic acid analogs such as peptide nucleic acids (PNA).

The term “replication module” as used herein refers to a consecutive sequence of nucleotides comprising at least one genetic element that is necessary to propagate a nucleic acid molecule comprising said consecutive sequence of nucleotides by producing at least one identical copy of said nucleic acid molecule in a living cell. Herein, the genetic element may include but is not limited to an “origin of replication” which is a particular sequence that is specifically recognized and bound by a protein complex in order to initiate the replication process.

The term “expression module” as used herein refers to a consecutive sequence of nucleotides comprising at least one genetic element that is suitable for performing a process by which information from a gene is used for the synthesis of a functional gene product in a living cell. Herein, the genetic element may include but is not limited to promoter and regulatory sequences.

The term “resistance module” as used herein refers to a consecutive sequence of nucleotides comprising at least one genetic element that is suitable for providing a living cell with resistance against a specific antibiotic.

The term “restriction site” as used herein refers to a consecutive sequence of nucleotides which is specifically recognized by a specific restriction enzyme that is able to cut a nucleic acid sequence between two nucleotides within said restriction site, or somewhere nearby.

The phrase “under stringent conditions” refers to conditions under which a nucleotide sequence will hybridize to a specific nucleic acid sequence, typically in a complex mixture of nucleic acids, but to essentially no other sequences. Stringent conditions are sequence-dependent and will be different in different circumstances.

Longer sequences hybridize specifically at higher temperatures. Generally, stringent conditions are selected to be about 5-10 degrees Celsius lower than the thermal melting point (Tm) for the specific sequence at a defined ionic strength pH. The Tm is the temperature (under defined ionic strength, pH, and nucleic acid concentration) at which 50% of the nucleotide sequence complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3, and the temperature is at least about 30 degrees Celsius for short sequences (e.g., 10 to 50 nucleotides) and at least about 60 degrees Celsius for long sequences (e.g., greater than 50 nucleotides). Stringent conditions may also be achieved with the addition of destabilizing agents such as formamide. Exemplary stringent hybridization conditions are often: 50% formamide, 5×SSC, and 1% SDS, incubating at 42 degrees Celsius, or, 5×SSC, 1% SDS, incubating at 65 degrees Celsius, with wash in 0.2×SSC, and 0.1% SDS at 65 degrees Celsius. Additional guidelines for determining hybridization parameters are provided in numerous references and are known by the person skilled in the art.

Methods to determine sequence identities between nucleic acid molecules are well known to a person skilled in the art and have been widely described, e.g., in US Patent Application 20140221623, which incorporated herein by reference in its entirety.

### Chemicals

Restriction enzymes, the Rapid DNA ligation kit, and Phusion DNA polymerase were purchased from Fermentas (Thermo Fisher Scientific, St. Leon-Rot, Germany). Gibson Assembly™ Master Mix was purchased from New England Biolabs (Ipswitch, USA).

### Molecular Genetics

The initial shuttle vector pPolyREP (GenBank acc. No. KF680544.1) was completely synthesized by Geneart® (Thermo Fisher Scientific, St. Leon-Rot, Germany). The first modification was the removal of the tandem histidin terminator sequences (his-Ter) by a PCR with 5′-phosphorylated primers (pPR-HisT_{—}1 & 2), followed by religation and transformation of E. coli DH5α. To rebuild the full-length vector starting from the isolated, truncated one, we conducted a Gibson Assembly [1] according to the manufacturer's manual with three amplificates. The first fragment was the original vector backbone with the newly generated terminator (new_Ter (SEQ ID NO: 3); primer pair GA_pPR_{—}1 & 2), while the other two fragments were the missing lacI gene (primer pair GA_pPR_{—}3 & 4) and the missing pBBR1 origin of replication (primer pair GA_pPR_{—}5 & 6), respectively. By the last two PCRs we also replaced the tandem rrnB terminator sequence (rrnR_ter) with a T7 terminator sequence (T7_ter; SEQ ID NO: 4) in the overlapping region of the fragments. And finally we modified the pWV01 origin of replication according to Bryksin & Matsumura (2010) by an amplification with the primer pairs pWV01_opt_{—}1 & 2 and insertion of the resulting 1,547 bp fragment in the shuttle vector via NotI, eventually yielding pPolyREPII. For expression trials, the gfp gene was amplified from pP_{T7}-GFP (MoBiTec GmbH, Göttingen, Germany) with the primer pair GFP_for (5′-phosphorylated) and GFP_His6_rev and ligated to pPolyREPII via EcoRV and XbaI. This cloned gene encodes GFP-His₆. All primer sequences mentioned here can be found in table 1. The correct sequences of all isolated vectors and inserts were confirmed by sequencing at MWG-Biotech AG (Ebersberg, Germany).

### Bacterial Strains, Transformation and Culture Conditions

Escherichia coli Stbl2 [2] was purchased from Life Technologies Corporation (Thermo Fisher Scientific, St. Leon-Rot, Germany) and used to harbor pPolyREP and derivatives. E. coli DH5α [3] was used as a host for pPolyREPII and derivatives. Pseudomonas putida KT2440 [4] and Bacillus subtilis 168 [5,6] were kindly provided by Prof. Dr. Susanne Fetzner (University of Münster, Germany). B. licheniformis DSM13 [7] and Cupriavidus metallidurans CH34 [8,9] were purchased from DSMZ (German Collection of Microorganisms and Cell Cultures, Braunschweig, Germany). The generation of competent cells, their transformation and selection with different concentrations of chloramphenicol is summarized in table 2. All strains harboring pPolyREP and pPolyREPII including their derivatives were grown in LB at 30° C. with the corresponding concentration of chloramphenicol. Autoinduction solutions M (50× stock: 1.25 M Na₂HPO₄, 1.25 M KH₂PO₄, 2.5 M NH₄Cl, 0.25 M Na₂SO₄) and 5052 (50× stock: 25% (v/v) glycerol, 2.5% (w/v) D-glucose, 10% (w/v) α-lactose monohydrate) [10] were added to induce the cells for expression of gfp. Four hours prior to cell harvest, the expression was additionally induced by the addition of 0.5 mM isopropyl β-D-1-thiogalactopyranoside (IPTG).

Structural and Segregational Stability and Copy Number of pPolyREPII

First, a starting culture of the plasmid-harboring strain was prepared. For this, the strain was grown over night in chloramphenicol-containing LB medium. Then, OD₆₀₀ was measured and fresh LB medium lacking chloramphenicol was inoculated with the starting culture to make an OD₆₀₀ of 0.010. The thus inoculated culture was grown for one day and then used to again to inoculate fresh LB medium. A total of five passages was done. At the start and after two and five passages, a 100-μl sample of the culture was withdrawn, adequately diluted and identical volumes of solution were plated on LB agar plates with and without chloramphenicol, respectively. After incubation, colonies obtained were counted and the ratio of cells that retained pPolyREPII was calculated. For each of the strains two independent experiments were performed. The plating of the cell suspensions was done in triplicates each.

To check the segregational stability of pPolyREPII, the above mentioned starting culture was used to inoculate fresh LB medium complemented with chloramphenicol to make a starting OD₆₀₀ of 0.010. After growth for one day, the culture was used to inoculate fresh chloramphenicol-containing LB medium. A total of five passages was done. The culture liquid of passage five was subjected to a plasmid isolation procedure. The kit-isolated plasmid DNA was digested with NotI and BglII and analyzed on an agarose gel. This experiment was performed twice independently for each of the strains.

To estimate the copy number of pPolyREPII, each of the strains was transformed with a reference plasmid with a known copy number. These strains and the corresponding pPolyREPII-harboring strains were grown overnight in LB medium with antibiotic and such pre-cultures were then used to inoculate fresh selective LB medium. Here, a starting OD₆₀₀ value of 0.010 was adjusted and cells were incubated overnight. After OD₆₀₀ measurement, cells were harvested (5500×g, 20 min, 10° C.) and the plasmid DNA (pDNA) was kit-isolated. DNA quantification was done using a NanoDrop™ 2000 (Thermo Scientific) and the pDNA was analyzed on an agarose gel. Based on quantification and comparison of band intensities using ImageJ (v1.48; open source; NIH, Bethesda, Md.) the pPolyREPII copy number in the different hosts was estimated. For this, above mentioned OD₆₀₀ values were taken into account, serving as a measure for the amount of starting cell material for pDNA isolation and, hence, for pDNA total yield calculation.

### Protein Analysis

Denaturing sodium dodecyl sulfate-polyacrylamide gel electrophoresis (SDS-PAGE) was performed as described by Laemmli [11], using an overall acrylamide concentration of 12% and a cross-linker concentration of 2.6% in the separating gels. Polyacrylamide gels were stained with Coomassie blue R-250 (0.1% (w/w) Coomassie blue R-250, 50% (w/w) trichloroacetic acid in H₂O) and destained in an aqueous solution of 30% (v/v) methanol and 10% (v/v) acetic acid. Transfer of proteins [12] from gels to nitrocellulose membranes (GE Healthcare Europe GmbH, Freiburg, Germany) was performed according to the protocol of QIAGEN (QIAexpress). Immunodetection of His₆-tagged proteins on blots was performed using Anti-Penta-His IgG1 HRP conjugate (Qiagen, Germany) and detection by chemiluminescence. GFP-His₆ synthesis in the crude extracts was visualized with a transilluminator (Blue LED Illuminator, excitation wavelength of 470 nm; NIPPON Genetics EUROPE GmbH, Germany).

Generation of the Initial Shuttle Vector pPolyREP

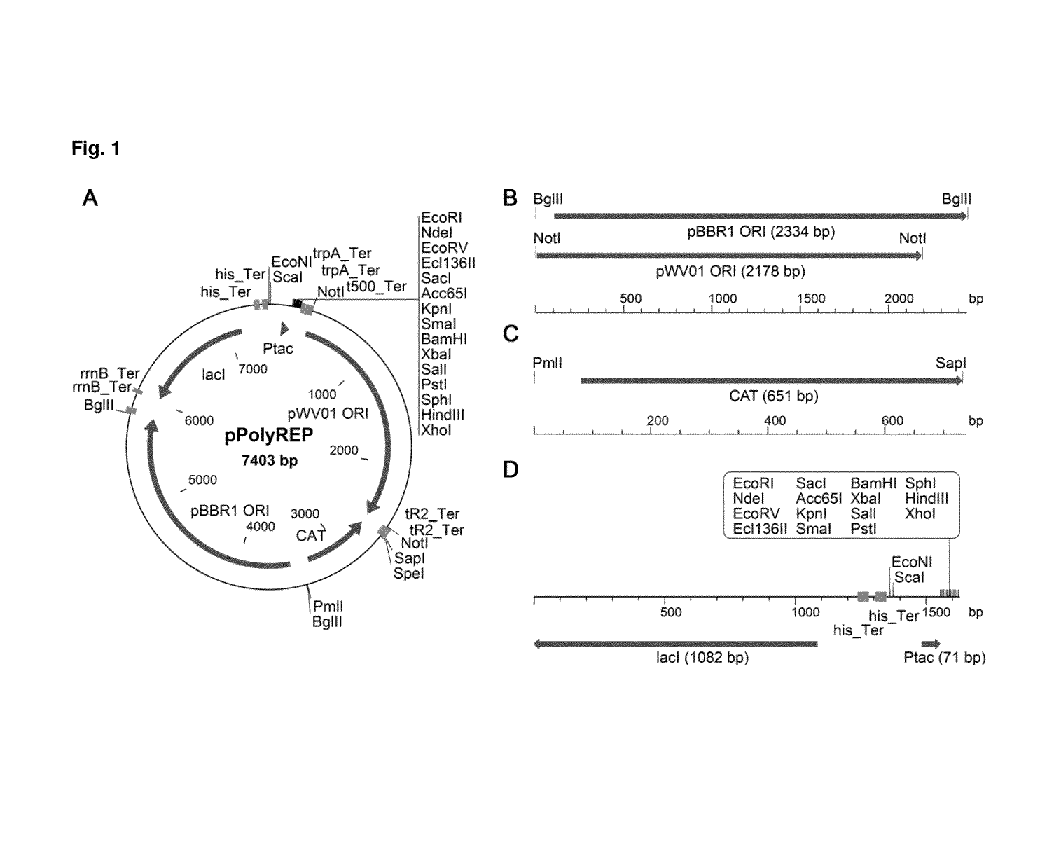

The shuttle vector was initially divided into functional subunits to facilitate the generation of defined modules that could be easily exchanged or deleted (FIG. 1). The first module was a replication module (FIG. 1B) based on the pBBR1 cassette, which is derived from Bordetella bronchiseptica S87. This origin of replication is known to replicate in a broad range of Gram(−) organisms [13,14] and is used in diverse vector systems. The second replication module was pWV01, which is derived from a cryptic plasmid found in Lactococcus lactis subsp. cremoris Wg2. Although this origin also replicates in E. coli, it predominantly replicates in Gram(+) organisms [31].

The third module was a chloramphenicol acetyl transferase (CAT) resistance cassette (FIG. 1C), comprising a common constitutive promoter followed by the CAT gene; this cassette can be found in many prokaryotes and thus is known to be functional in many different hosts [17,18].

The fourth module was the expression module (FIG. 1D), comprising a lacI cassette and an improved hybrid of the common lac UV5 promoter (Plac) and trp promoter (Ptrp) known as Ptac [19] (SEQ ID NO: 7). This was followed by a lac operator and a multiple cloning site. The Ptac promoter is approximately seven times stronger than the common Plac promoter, but is still compatible with a broad range of Gram(+) and Gram(−) hosts.

The introduction of transcription terminators can promote vector stability in different hosts [15,20]. Therefore, we introduced a series of different transcription terminators (for an overview see: [21]) downstream of each gene and the multiple cloning site. The complete synthesis and assembly of the full-length shuttle vector was carried out by GeneArt® (Thermo Fisher Scientific, St. Leon-Rot, Germany).

Evaluation of pPolyREP

The initial shuttle vector pPolyREP was propagated solely by the specialized E. coli strain Stbl2, which unlike the common strain DH5α can maintain DNA sequences containing unstable repeats [2]. A gfp gene with a downstream His₆ tag sequence was cloned in this vector, generating pPR::GFP-His₆. E. coli Stbl2, B. subtilis 168, B. licheniformis DSM13, C. metaffidurans CH34 and P. putida KT2440 were each transformed separately with pPolyREP and pPR::GFP-His₆.

Although it was possible to select resistant transformants for all strains, GFP-His₆ was only synthesized in E. coli Stbl2 and P. putida KT2440 (data not shown). This suggested that the other strains may have experienced problems similar to those reported in E. coli DH5α, namely, the truncation of the plasmid, reflecting the presence of multiple terminator sequences. It was also unclear whether pWV01 was functional, because only Gram(−) organisms were able to synthesize GFP-His₆.

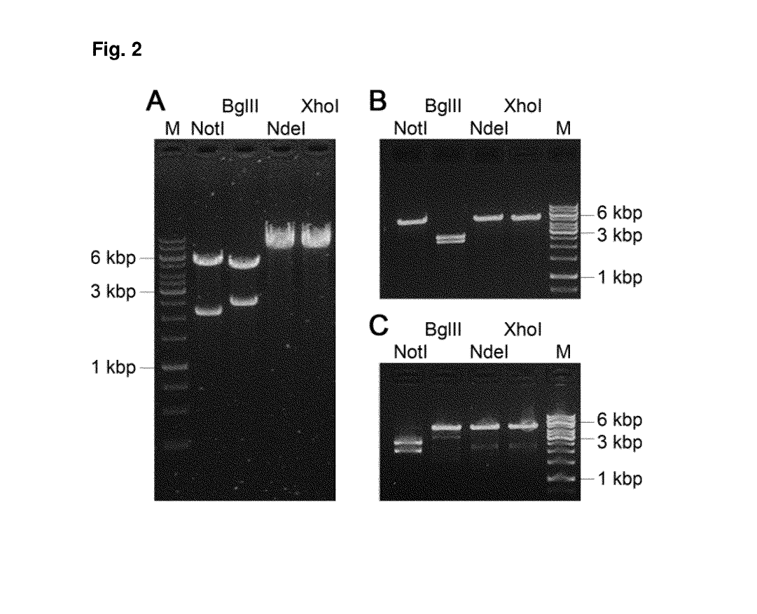

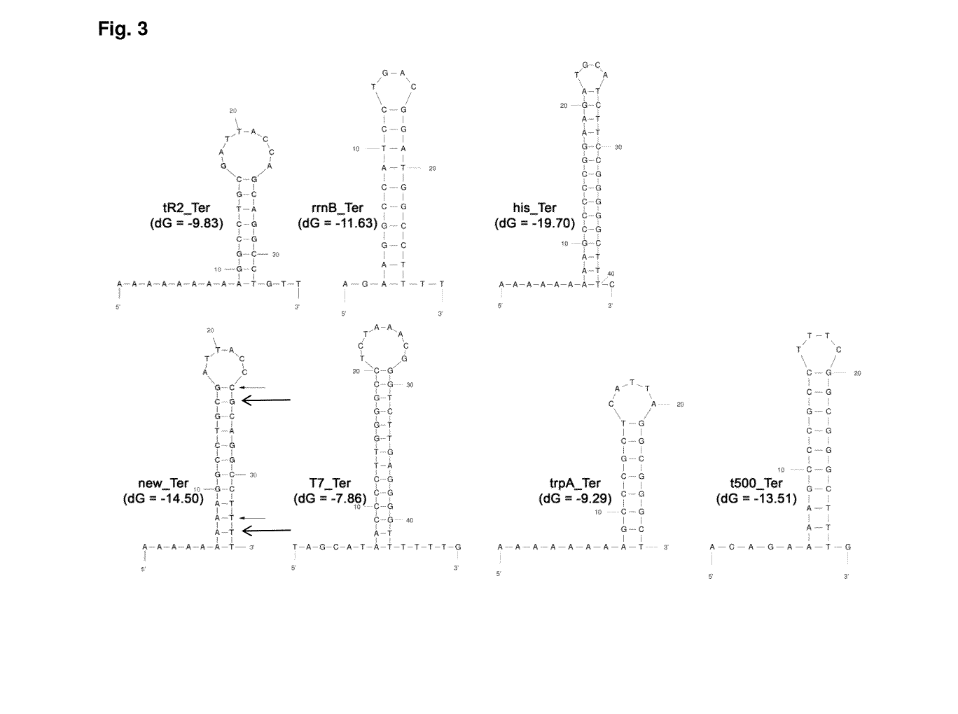

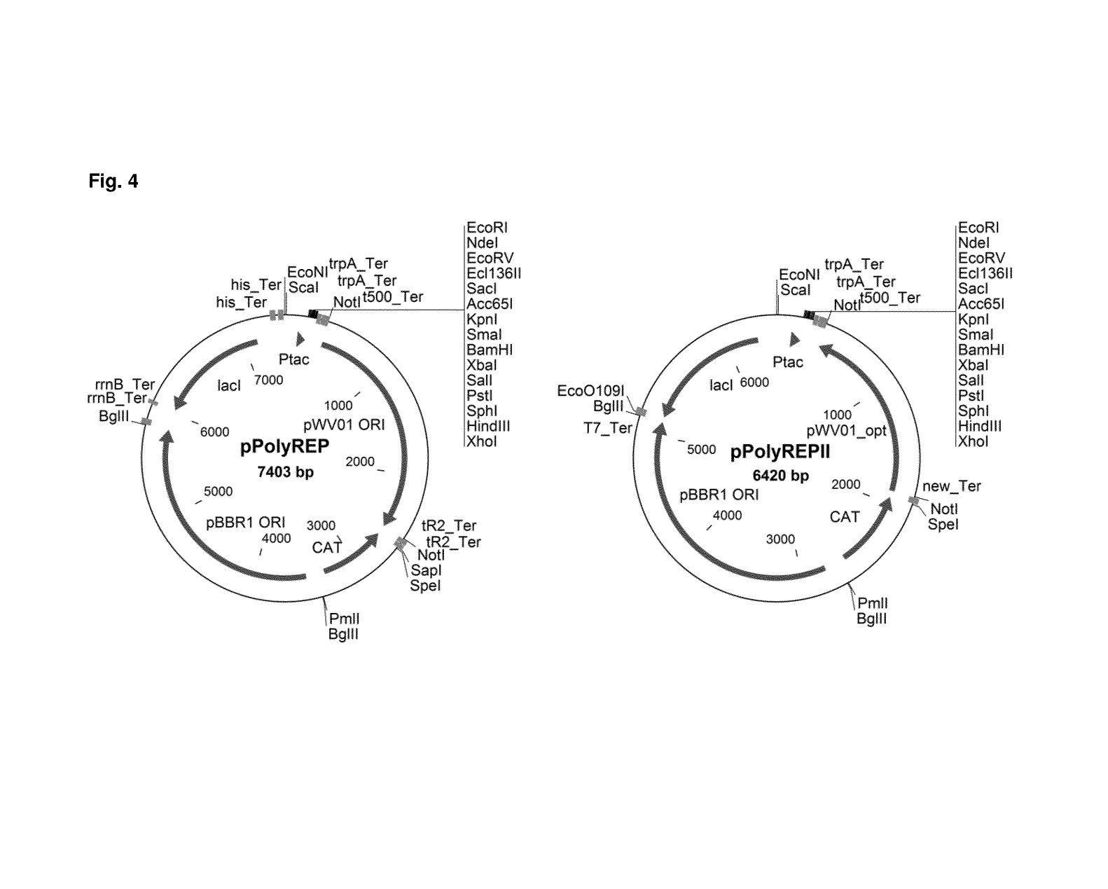

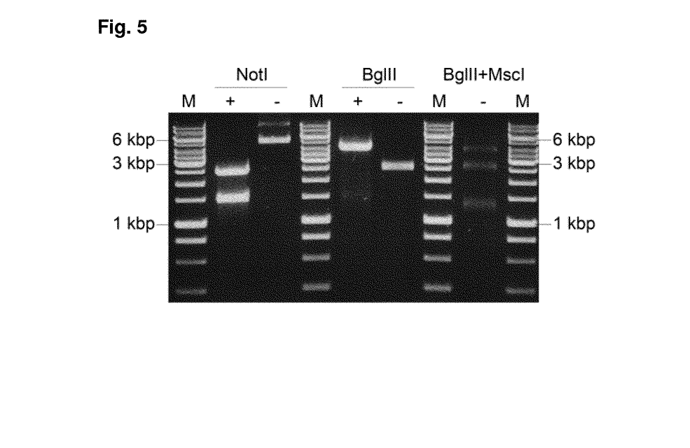

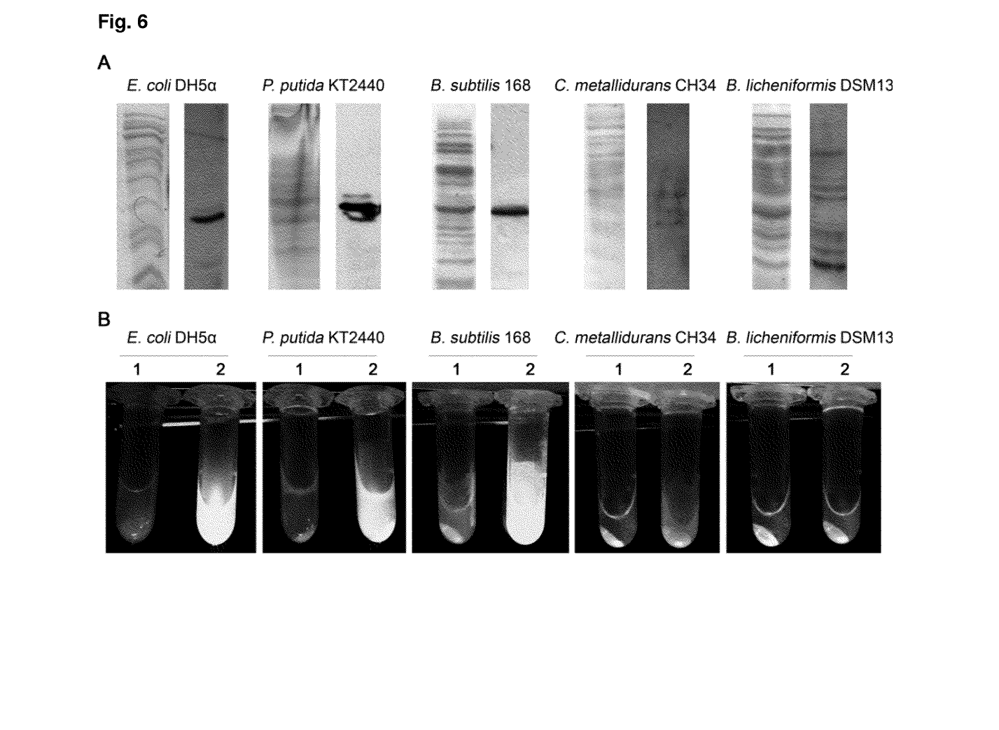

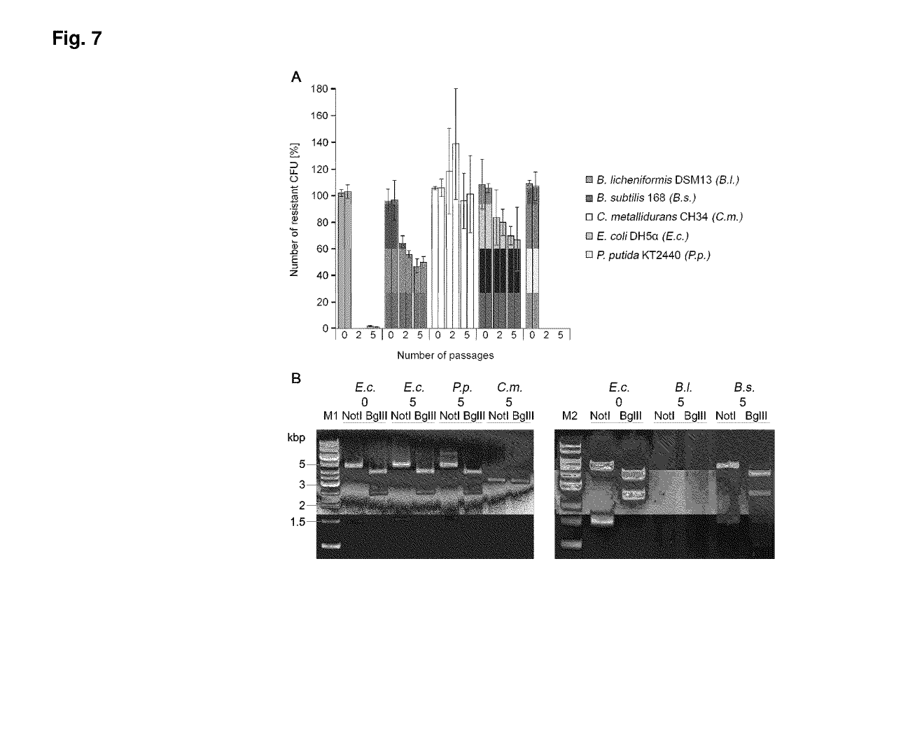

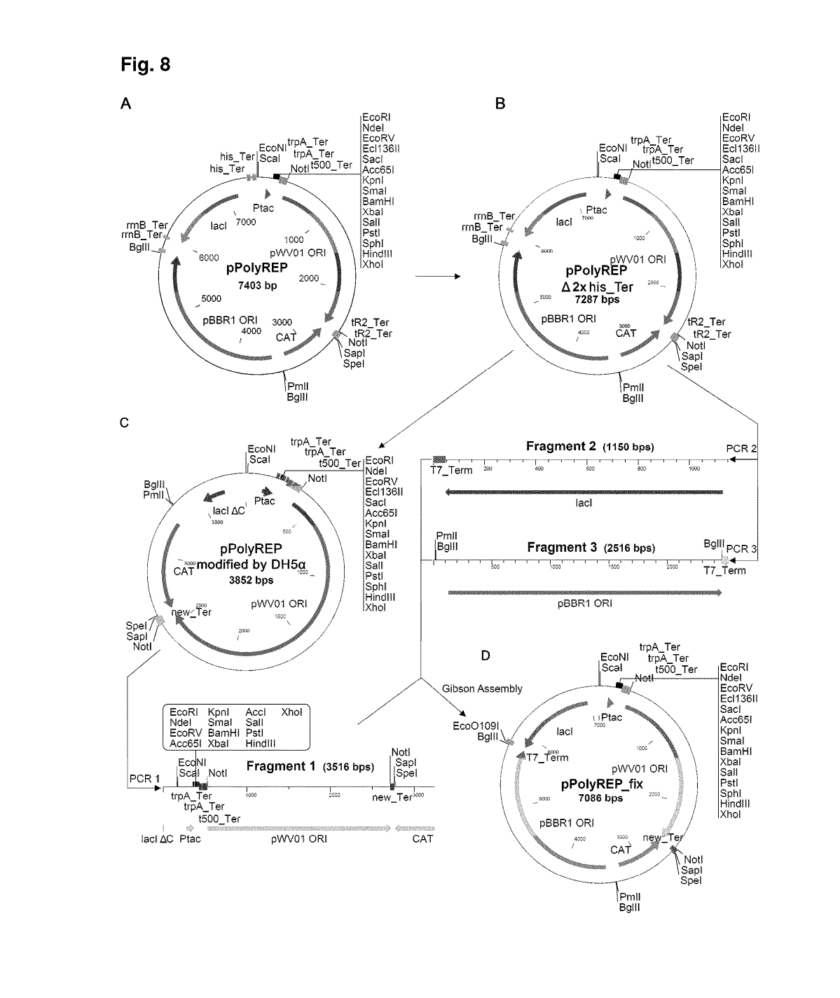

We therefore exploited the modular design of the shuttle vector and removed either of the two origins of replication by cutting the vector with BglII or NotI, generating pPR_pBBR1 and pPR_pWV01, respectively (FIG. 2). We transformed E. coli Stbl2 with both of these vectors, and plasmid DNA isolated from the transformants revealed the vectors were recovered unchanged, with single origins as anticipated (FIGS. 2B&C). In contrast to B. licheniformis DSM13, B. subtilis 168 and C. metallidurans CH34, the structural stability of pPolyREP in E. coli Stbl2 and P. putida KT2440 seems to be high, since a synthesis of GFP was shown and intact plasmids were isolated from both (results for P. putida KT2440 not shown).

Optimization of pPolyREP to pPolyREPII

Our initial hypothesis explaining the failure of the shuttle vector to replicate in common E. coli strains was the large number and tandem organization of the terminator sequences. Analysis of the terminator regions in pPolyREP using the mfold program [22] showed that the tandem histidine terminators (his_Ter) and the tandem tryptophan terminators (trpA_Ter; SEQ ID NO: 5) were the strongest terminator regions, based on their calculated high Gibbs free energy values [23]. We therefore used PCR to delete the corresponding regions from pPolyREP and introduced the derived vectors into E. coli DH5α, which was unable to maintain the original vector. We recovered transformants containing the vector derivative with the histidine terminator sequences deleted but the tryptophan terminators remaining intact, indicating that the host experienced difficulty with the tandem histidine terminators. However the sequence of the isolated vector revealed additional truncations in the region between lacI and pBBR1, suggesting that the tandem rrnB terminators were also problematic. Interestingly, the tandem tR2 terminators (tR2_Ter) were also found to be modified by the partial deletion of one copy and the introduction of two point mutations. This generated a completely new terminator (new_Ter; SEQ ID NO: 3) as confirmed with the mfold program (FIG. 3). The initial set of terminator sequences of pPolyREP is shown in FIG. 3A, while the optimized set of terminator sequences found in pPolyREPII is depicted in FIG. 3B. To complement the missing parts, we carried out a Gibson assembly with three parts, directly replacing the dual rrnB terminator with a T7 terminator (T7_Ter; SEQ ID NO: 4). The resulting vector was maintained in E. coli DH5α at full length. To prevent any problems that might be caused by a potentially unstable pWV01 ori, we also truncated the sequence involved in the copy number control mechanism as described by Bryksin and Matsumura [16]. The pWV01 origin of replication may be unstable in heterologous hosts since the copy number control mechanism may outweigh the RCR. The optimized ori, pWV01_opt, was inserted in opposite orientation via the NotI site, yielding the final shuttle vector, pPolyREPII (FIG. 4). The whole optimization procedure from pPolyREP to pPolyREPII is depicted in FIG. 8.

Evaluation of pPolyREPII

The full-length vector pPolyREPII was digested with BglII or NotI to generate the derivatives pPolyREPII(+) (pPRII(+)) and pPolyREPII(−) (pPRII(−)), which carry only the pWV01 or pBBR1 origins, respectively. Digesting these derivatives with BglII or NotI or BglII and MscI produced fragments of the anticipated sizes (FIG. 5). We also sequenced pPolyREPII and both derivatives to verify the expected sequences. The expression capabilities of pPolyREPII were evaluated by inserting a gfp gene with a downstream His₆ tag sequence, generating pPRII::GFP-His₆. E. coli Stbl2, B. subtilis 168, B. licheniformis DSM13, C. metallidurans CH34 and P. putida KT2440 were each transformed separately with pPolyREPII, pPRII(+), pPRII(−) and pPRII::GFP-His₆. The corresponding transformation efficiencies are listed in Table 3. Following induction of the clones bearing pPolyREPII as the empty vector control and pPRII::GFP-His₆, crude extracts from each strain were analyzed by SDS-PAGE, western blot and GFP fluorescence (FIG. 6). We observed strong GFP expression in E. coli DH5α, Pseudomonas putida KT2440, Bacillus subtilis 168, weak expression in Cupriavidus metallidurans CH34, and no expression in Bacillus licheniformis DSM13. The presence of the GFP-His₆ protein was confirmed by western blot. Although we did not see an expression of gfp in B. licheniformis DSM13, it must not point to a failed transformation, especially because we were able to differentiate resistant transformats from those which were not resistant. However a problem may be based on the expression reporter, gfp itself, since it is the unmodified “wildtype” form, which is not optimized for expression in any of those hosts. This can be seen for the expression in C. metallidurans CH34, which is much weaker than that in the other hosts.

To further characterize pPolyREPII, we analyzed both its segregational and structural stability (FIG. 7). Results obtained revealed a significant host dependency regarding the vector's segregational stability. Virtually no loss was observed for C. metallidurans CH34. In E. coli DH5α and B. subtilis 168 a moderate reduction of plasmid-harboring cells was detected over time (FIG. 7A), i.e., after 5 passages of the former strain about 70% of the cells retained pPolyREPII. For the latter strain a value of approx. 50% was observed. For both B. licheniformis DSM13 and P. putida KT2440, a complete loss of the plasmid, already after 2 passages, was detected. There is no correlation for this behavior with respect of the used origin of replication, since both the strictly pBBR1-dependent strains (C. metaffidurans CH34 and P. putida KT2440) as well as the strictly pWV01-dependent strains (B. subtilis 168 and B. licheniformis DSM13) have segregational stable and unstable representatives. However we were still able to isolate intact pPolyREPII from E. coli DH5α, P. putida KT2440 and B. subtilis 168 after 5 rounds of inoculation as indicated by restriction patterns after NotI and BglI digestion, respectively (FIG. 7B). The restriction pattern of digested vector DNA from C. metaffidurans CH34 resulted in a single band corresponding to the linearized vector, possibly indicating an incomplete digestion as also slightly seen at a corresponding band in the lane for P. putida KT2440.

To estimate the copy number of pPolyREPII in the hosts tested, they were transformed with established vectors whose copy numbers are known and compared the yield of vector DNA isolated from a culture of the same OD_{600 nm}. It was possible to estimate the copy numbers of pPolyREPII in all hosts tested except for B. licheniformis DSM13 due to isolation problems of vector DNA (Table 4).

As a result, a very effective broad host range expression vector is provided, which can be equally well established in Gram(+) and Gram(−) hosts. This is possible by a rational design and using the recent advantages in synthetic biology. The novel nucleic acid molecule (expression vector) according to the invention can be used to establish eDNA expression libraries and to screen for desired activities in different Gram(+) as well as Gram(−) hosts at the same time.

TABLE 1
Primer sequences used to generate
pPolyREPII and derivatives.

| Primer | Sequence (5′→3′) |
|---|---|
| pPR-HisT_1 | PHO-CACCGTGCAGTCGATAAGC |
| | (SEQ ID NO: 12) |
| pPR-HisT_2 | PHO-GCTGTGGTATGGCCTGTG |
| | (SEQ ID NO: 13) |
| GA_pPR_1 | CACATTCACCACCCTGAATTGAC |
| | (SEQ ID NO: 14) |
| GA_pPR_2 | GATCTCACGTGGGATTGATTCTAATG |
| | (SEQ ID NO: 15) |
| GA_pPR_3 | TAGCATAACCCCTTGGGGCCTCTAAACGGGTCTTGA |
| | GGGGTTTTTTGTCACTGCCCGCTT |
| | (SEQ ID NO: 16) |
| GA_pPR_4 | GTCAATTCAGGGTGGTGAATGTG |
| | (SEQ ID NO: 17) |
| GA_pPR_5 | CATTAGAATCAATCCCACGTGAGATC |
| | (SEQ ID NO: 18) |
| GA_pPR_6 | CAAAAAACCCCTCAAGACCCGTTTAGAGGCCCCAAGG |
| | GGTTATGCTAAGATCTATCGCCC |
| | (SEQ ID NO: 19) |
| pWV01_opt_1 | AAGGAAAAAAGCGGCCGCCGATTTTTTATTAAAAC |
| | GTCTCAAAATCGTTTCTGAG (SEQ ID NO: 20) |
| pWV01_opt_2 | AAGGAAAAAAGCGGCCGCGTCATTTTATTTCCCC |
| | CGTTTCAGCATC (SEQ ID NO: 21) |
| GFP_for | PHO-ATGGTCCAAACTAGTTCGAAGATC |
| | (SEQ ID NO: 22) |
| GFP_His6_rev | GCTCTAGACTAATGATGATGGTGATGATGTTTGTA |
| | GGGCTCATCCATGC (SEQ ID NO: 23) |

TABLE 2
Transformation and selection of each strain. After
transformation each strain was regenerated with SOC
medium (Life Technologies Corporation) for 2 h at 30°
C. before plating out on LB selection agar.

| | Method of | Reference for | Chloramphenicol |
|---|---|---|---|
| Strain | transformation | transformation | (μg/ml) |
| B. subtilis 168 | Protoplasts | [28] | 5 |
| B. licheniformis | Electroporation | [24] | 15 |
| DSM13 | | | |
| C. metallidurans | Electroporation | [25] | 150 |
| CH34 | | | |
| E. coli Stbl2 | Heat shock | [26] | 10 |
| E. coli DH5α | Heat shock | [26] | 10 |
| P. putida | Electroporation | [27] | 250 |
| KT2440 | | | |

TABLE 3
Transformation efficiency of pPolyREPII, pPRII::GFP-His6,
pPRII(+), and pPRII(−) in various hosts.

| | Transformation | | | | |
|---|---|---|---|---|---|
| | efficiency | | pPRII::GFP- | | |
| Recipient strain | [cfu/μg DNA] | pPolyREPII | His₆ | pPRII(+) | pPRII(−) |
| E. coli DH5α | Minimum | 1.3 × 10⁴ | 8.8 × 10² | 2.8 × 10³ | 6.9 × 10³ |
| | Maximum | 1.3 × 10⁵ | 6.2 × 10⁴ | 4.1 × 10⁴ | 6.3 × 10⁴ |
| P. putida | Minimum | 1.5 × 10² | 1.1 × 10² | NT | 8.0 × 10² |
| KT2440 | Maximum | 3.4 × 10³ | 6.5 × 10³ | NT | 9.5 × 10³ |
| C. metallidurans | Minimum | 9.7 × 10² | 1.1 × 10² | NT | 7.5 × 10² |
| CH34 | Maximum | 2.9 × 10⁴ | 1.5 × 10⁴ | NT | 5.9 × 10⁴ |
| B. subtilis 168 | Minimum | 7.8 × 10³ | 2.3 × 10³ | 5.7 × 10³ | NT |
| | Maximum | 8.2 × 10³ | 3.2 × 10³ | 7.7 × 10³ | NT |
| B. licheniformis | Minimum | 4.1 × 10¹ | 7.7 × 10¹ | 1.1 × 10² | NT |
| DSM13 | Maximum | 5.2 × 10¹ | 1.3 × 10² | 1.3 × 10² | NT |

NT = no transformants detected. Transformation of B. licheniformis DSM13 with methylated pDNA resulted in an increased transformation efficiency of 2-4 folds. The transformation efficiencies were determined in two independent experiments.

TABLE 4
Estimated copy numbers of pPolyREPII in various hosts as compared
with literature derived copy numbers of established vectors.

| | Estimated | Compared with |
|---|---|---|
| Recipient strain | copy number | (copy number) |
| E. coli DH5α | 30 | pET-44a(+) |
| | | (~40) |
| P. putida | 2-3 | pBBR1MCS-2 |
| KT2440 | | (4-10) |
| C. metallidurans | 1-2 | pBBR1MCS-1 |
| CH34 | | (4-10) |
| B. subtilis 168 | 9 | pUMB |
| | | (46-50) |
| B. licheniformis | n.d. | — |
| DSM13 | | |

n.d., not determined due to isolation problems of the vector.

### LITERATURE

1. Gibson D, Young L, Chuang R, Venter J, Hutchison III C, et al. (2009) Enzymatic assembly of DNA molecules up to several hundred kilobases. Nature Methods 6: 343-345.
2. Trinh T, Jessee J, Bloom F, Hirsch V (1994) STBL2: an Escherichia coli strain for the stable propagation of retroviral clones and direct repeat sequences. Focus 16: 78-80.
3. Grant S G, Jessee J, Bloom F R, Hanahan D (1990) Differential plasmid rescue from transgenic mouse DNAs into Escherichia coli methylation-restriction mutants. Proceedings of the National Academy of Sciences of the United States of America 87: 4645-4649.
4. Nelson K E, Weinel C, Paulsen I T, Dodson R J, Hilbert H, et al. (2002) Complete genome sequence and comparative analysis of the metabolically versatile Pseudomonas putida KT2440. Environmental microbiology 4: 799-808.
5. Zeigler D R, Prágai Z, Rodriguez S, Chevreux B, Muffler A, et al. (2008) The origins of 168, W23, and other Bacillus subtilis legacy strains. Journal of bacteriology 190: 6983-6995.
6. Barbe V, Cruveiller S, Kunst F, Lenoble P, Meurice G, et al. (2009) From a consortium sequence to a unified sequence: the Bacillus subtilis 168 reference genome a decade later. Microbiology (Reading, England) 155: 1758-1775.
7. Veith B, Herzberg C, Steckel S, Feesche J, Maurer K H, et al. (2004) The complete genome sequence of Bacillus licheniformis DSM13, an organism with great industrial potential. Journal of molecular microbiology and biotechnology 7: 204-211.
8. Vandamme P, Coenye T (2004) Taxonomy of the genus Cupriavidus: a tale of lost and found. International journal of systematic and evolutionary microbiology 54: 2285-2289.
9. Vaneechoutte M (2004) Wautersia gen. nov., a novel genus accommodating the phylogenetic lineage including Ralstonia eutropha and related species, and proposal of Ralstonia [Pseudomonas] syzygii (Roberts et al. 1990) comb. nov. International Journal of Systematic and Evolutionary Microbiology 54: 317-327.
10. Studier W F (2005) Protein Production by Auto-Induction in High-Density Shaking Cultures. Protein Expression and Purification 41: 207-234.
11. Laemmli U (1970) Cleavage of structural proteins during the assembly of the head of bacteriophage T4. Nature 227: 680-685.
12. Towbin H, Staehelin T, Gordon J (1979) Electrophoretic transfer of proteins from polyacrylamide gels to nitrocellulose sheets: procedure and some applications. 1979. Proceedings of the National Academy of Sciences of the United States of America 76: 4350-4354.
13. Prior J E, Lynch M D, Gill R T (2010) Broad-host-range vectors for protein expression across gram negative hosts. Biotechnology and bioengineering 106: 326-332.
14. Lale R, Brautaset T, Valla S (2011) Broad-Host-Range Plasmid Vectors for Gene Expression in Bacteria. In: Williams J A, editor. Strain Engineering SE—19. Humana Press, Vol. 765. pp. 327-343.
15. Pérez-arellano I, Zúñiga M, Pérez-Martínez G (2001) Construction of compatible wide-host-range shuttle vectors for lactic acid bacteria and Escherichia coli. Plasmid 46: 106-116.
16. Bryksin A V, Matsumura I (2010) Rational design of a plasmid origin that replicates efficiently in both gram-positive and gram-negative bacteria. PloS one 5: e13244.
17. Schwarz S, Kehrenberg C, Doublet B, Cloeckaert A (2004) Molecular basis of bacterial resistance to chloramphenicol and florfenicol. FEMS microbiology reviews 28: 519-542.
18. Murray I A, Shaw W V (1997) 0-Acetyltransferases for Chloramphenicol and Other Natural Products. Antimicrobial Agents and Chemotherapy 41: 1-6.
19. Comstock J, de Boer H A, Comstock L J, Vasser M (1983) The tac promoter: A functional hybrid derived from the trp and lac promoters. Biochemistry 80: 21-25.
20. Kieser T, Melton R E (1988) Plasmid pIJ699, a multi-copy positive-selection vector for Streptomyces. Gene 65: 83-91.
21. Larson M H, Greenleaf W J, Landick R, Block S M, Program B (2008) Applied force reveals mechanistic and energetic details of transcription termination. Cell 132: 971-982.
22. Zuker M (2003) Mfold web server for nucleic acid folding and hybridization prediction. Nucleic Acids Research 31: 3406-3415.
23. SantaLucia J (1998) A unified view of polymer, dumbbell, and oligonucleotide DNA nearest-neighbor thermodynamics. Proceedings of the National Academy of Sciences of the United States of America 95: 1460-1465.
24. Xue G-P, Johnson J S, Dalrymple B P (1999) High osmolarity improves the electro-transformation efficiency of the gram-positive bacteria Bacillus subtilis and Bacillus licheniformis. Journal of Microbiological Methods 34: 183-191.
25. Taghavi S, van der Lelia D, Mergeay M (1994) Electroporation of Alcaligenes eutrophus with (Mega) Plasmids and Genomic DNA Fragments. Applied and Environmental Microbiology 60: 3585-3591.
26. Hanahan D (1983) Studies on transformation of Escherichia coli with plasmids. Journal of Molecular Biology 166: 557-580.
27. Iwasaki K, Uchiyama H, Yagi O, Kurabayashi T, Ishizuka K, et al. (1994) Transformation of Pseudomonas putida by Electroporation. Bioscience, Biotechnology, and Biochemistry 58: 851-854.
28. Degering C, Eggert T, Puls M, Bongaerts J, Evers S, et al. (2010) Optimization of protease secretion in Bacillus subtilis and Bacillus licheniformis by screening of homologous and heterologous signal peptides. Appl Environ Microbiol 76: 6370-6376.

What is claimed:

1. A synthetic nucleic acid molecule for expressing at least one nucleotide sequence of interest in at least one prokaryotic host cell, comprising:
at least one promoter sequence,
at least one cloning site for inserting the nucleotide sequence of interest, wherein the cloning site is located downstream of the promoter sequence, and
at least one replication module comprising:
at least one replication cassette for promoting replication of the nucleic acid molecule in Gram-negative organisms, and
at least one replication cassette for promoting replication of the nucleic acid molecule in Gram-positive organisms,
at least one expression module for promoting expression of the nucleotide sequence of interest in the host cell, and
at least one resistance module for providing the host cell with antibiotic resistance,
wherein the at least one replication module, the at least one expression module and the at least one resistance module are each flanked at both ends by at least one unique restriction site.

2. The nucleic acid molecule according to claim 1, wherein the replication cassette for Gram-negative organisms comprises a pBBR1 origin of replication.

3. The nucleic acid molecule according to claim 2, wherein the replication cassette for Gram-negative organisms comprises the nucleotide sequence according to SEQ ID NO: 1.

4. The nucleic acid molecule according to claim 1, wherein the replication cassette for Gram-positive organisms comprises a pWV01 origin of replication.

5. The nucleic acid molecule according to claim 4, wherein the pWV01 origin of replication is a modified pWV01 origin of replication.

6. The nucleic acid molecule according to claim 4, wherein the replication cassette for Gram-positive organisms comprises the nucleotide sequence according to SEQ ID NO: 2.

7. The nucleic acid molecule according to claim 1, wherein the unique restriction site is selected from the group consisting of BglII, NotI, PmlI, and SapI.

8. The nucleic acid molecule according to claim 1, further comprising at least one transcription termination sequence located downstream of the cloning site, preferably selected from the group consisting of SEQ ID NO: 3, SEQ ID NO: 4, SEQ ID NO: 5, and SEQ ID NO: 6.

9. The synthetic nucleic acid molecule according to claim 1, comprising at least one nucleotide sequence selected from the group consisting of:
a) a nucleotide sequence which comprises the nucleotide sequences of SEQ ID NO: 1, SEQ ID NO: 2, SEQ ID NO: 3, SEQ ID NO: 4, SEQ ID NO: 5, SEQ ID NO: 6, or SEQ ID NO: 7;
b) a nucleotide sequence according to SEQ ID NO: 8;
c) a nucleotide sequence, the complementary strand of which hybridizes with the nucleotide sequences of a) or b) under stringent conditions;
d) a nucleotide sequence which has at least 90%, preferably 95%, identity with the nucleotide sequence of a), b) or c); and
e) a nucleotide sequence which corresponds to the complementary strand of the nucleotide sequence of a) to d).

10. A prokaryotic cell including the nucleic acid molecule according to claim 1.

11. A cell culture comprising at least one cell according to claim 10.

12. Method for expressing at least one nucleotide sequence of interest in at least one prokaryotic host cell, comprising:
inserting the nucleotide sequence of interest into the cloning site of the nucleic acid molecule according to claim 1;
subsequently, introducing the nucleic acid molecule into a host cell to obtain a modified host cell; and
cultivating the modified host cell under conditions that allow expression of the nucleotide sequence of interest.

13. The method of claim 12, wherein the nucleic acid molecule is heterologously expressed in at least one prokaryotic host cell.

14. Method according to claim 12, wherein the nucleotide sequence of interest is part of a metagenomic library.

15. The method of claim 14, wherein the metagenomic library is an environmental expression library and is functionally screened.

16. Method for producing a shuttle vector comprising:
providing:
(i) at least one replication module comprising:
at least one replication cassette for promoting replication of a nucleic acid molecule in Gram-negative organisms, and
at least one replication cassette for promoting replication of a nucleic acid molecule in Gram-positive organisms,
(ii) at least one expression module for promoting expression of a nucleotide sequence of interest in a host cell, and
(iii) at least one resistance module for providing the host cell with antibiotic resistance,
and assembling (i), (ii) and (iii) to obtain said shuttle vector.
